(19) United States
(12) Patent Application Publication
(10) Pub. No.: US 2023/0113074 A1
(43) Pub. Date: Apr. 13, 2023

(54) DERIVATION OF HEPATOCYTES AND HEMATOPOIETIC PROGENITORS FROM HUMAN EMBRYONIC STEM CELLS

(71) Applicant: Wisconsin Alumni Research Foundation, Madison, WI (US)

(72) Inventors: Srikumar Sengupta, Madison, WI (US); James Thomson, Madison, WI (US)

(21) Appl. No.: 17/963,806

(22) Filed: Oct. 11, 2022

Related U.S. Application Data
(60) Provisional application No. 63/254,830, filed on Oct. 12, 2021.

(51) Int. Cl. C12N 5/0735

(52) U.S. Cl. CPC C12N 5/0606; C12N 2501/155; C12N 2501/119; C12N 2506/02; C12N 2501/16

(57) ABSTRACT

This disclosure relates generally to methods for generating small hepatocyte progenitor cells (SHPCs) and hematopoietic progenitor cells (HPCs) from human embryonic stem cells, and hematopoietic progenitor cells from primary human endothelial cells and cell lines populations of small hepatocyte progenitor cells and hematopoietic progenitor cells, and uses thereof.

### FIELD OF DISCLOSURE

This disclosure relates generally to methods for generating small hepatocyte progenitor cells and hematopoietic progenitor cells from human embryonic stem cells, hematopoietic progenitor cells from primary human endothelial cells and cell lines, populations of small hepatocyte progenitor cells and hematopoietic progenitor cells, and uses thereof.

### BACKGROUND

Chronic liver disease is a leading cause of death in the United States. Generation of small hepatocyte progenitor cells (SHPCs) could help aid in the treatment of inherited and acquired liver diseases. However, current SHPCs generated from primary human hepatocytes (PPHs) have limited passage rates. Derivation and culture of SHPCs capable of proliferating in vitro has been described in rodents and recently in humans. These cells are capable of engrafting in injured livers in initial passages, however, they display dedifferentiated morphology, reduced xenobiotic metabolism activity in culture and loss of engraftment over passages.

Studies in rats have demonstrated that the restoration of tissue mass in injured livers can be achieved through either proliferation of mature hepatocytes or, under impaired cell division, through the expansion of a population of SHPCs. SHPCs have also been successfully isolated and grown ex vivo from hepatocytes obtained from adult rat livers. Recently, SHPCs have also been derived from PHHs that show engraftment in mouse livers and are capable of supporting human hepatitis B virus infection, however these cells display diminished metabolic activity over passages. Human SHPCs have been shown to reconvert to a metabolically active state after treatment with specialized conditions, but these cells can only be derived from PHHs obtained from infant livers. We have previously shown generation of SHPCs from adult liver-derived PHHs that maintain metabolic maturity over many passages (PMID: 34345838). SHPCs are however difficult to generate from hepatocytes obtained from diseased livers and hence there needs to be an alternate approach for generating these cells for transplantation in the clinic.

Human stem cell/induced pluripotent stem cell (ES/iPS)-derived SHPCs offer an attractive alternative to PHHs for autologous transplantation. Further, ES/iPS-derived SHPCs would be invaluable for drug toxicity studies, as PHHs are limited in supply, vary in their metabolic activity between donors, and may lack the quality needed for generation of SHPCs.

There are currently no pluripotent stem cell-derived hepatocytes or hematopoietic progenitor cells that engraft in immunodeficient mice. The discovery of embryonic stem cells (ES)-derived mature hematopoietic progenitor cells (HPCs) would allow for treatment of multiple hematologic malignancies. Further, patient specific endothelial cell-derived engraftable HPCs would also allow autologous transplantation without the need for immunosuppression for treatment of hematopoietic malignancies. Given how essential SHPCs and HPCs are, there is a need in the art for an improved and standardized method of producing engraftable mature hematopoietic progenitor cells or hepatocytes for drug and toxicity testing, treatments for liver and hematopoietic diseases and virus studies. SHPCs would be invaluable for study of hepatitis viruses (HBV, HCV) and HPC derivatives would be useful for studies of retroviruses (HIV, HTLV and XMRV).

### SUMMARY OF THE DISCLOSURE

Provided herein is a method for obtaining small hepatocyte progenitor cells and hematopoietic progenitor cells, the method comprising: culturing human embryonic stem cells in the presence of embryonic fibroblasts in a culture medium that comprises at least one of Activin A, a bone morphogenetic protein, a fibroblast growth factor, a TGF-beta inhibitor, a notch pathway inhibitor, a hepatocyte growth factor, whereby a cell population comprising small hepatocyte progenitor cells and hematopoietic progenitor cells is obtained.

Also provided herein is a method of obtaining a substantially pure, isolated population of small hepatocyte progenitor cells and CD45+ hematopoietic progenitor cells, the method comprising: culturing human embryonic stem cells in the presence of embryonic fibroblasts in a culture medium that comprises at least one of Activin A, a bone morphogenetic protein, a fibroblast growth factor, a TGF-beta inhibitor, a notch pathway inhibitor, a hepatocyte growth factor, whereby a cell population comprising small hepatocyte progenitor cells and hematopoietic progenitor cells is obtained.

Also provided herein is a pharmaceutical composition comprising a substantially pure, isolated population of small hepatocyte progenitor cells and CD45+ hematopoietic progenitor cells, and a pharmaceutically acceptable carrier.

Also provided herein is a method for obtaining hematopoietic progenitor cells, the method comprising: culturing human primary endothelial cells in the presence of embryonic fibroblasts in a culture medium whereby a cell population comprising CD45+ hematopoietic progenitor cells is obtained.

Also provided herein is a method of obtaining a substantially pure, isolated cell population of CD45+ hematopoietic progenitor cells, the method the method comprising: culturing human primary endothelial cells in the presence of embryonic fibroblasts in a culture medium whereby a cell population comprising CD45+ hematopoietic progenitor cells is obtained.

Also provided herein is a pharmaceutical composition comprising a substantially pure, isolated cell population of CD45+ hematopoietic progenitor cells and a pharmaceutically acceptable carrier.

Also provided herein is a method for obtaining a population of macrophages, the method comprising: culturing the population of comprising CD45+ hematopoietic progenitor cells obtained according to the method of claim 13 in absence of a matrix in a culture medium and under conditions suitable for generation of macrophages, whereby a cell population comprising macrophages is obtained.

Also provided herein is a cell culture medium comprising Activin A, a bone morphogenetic protein, a fibroblast growth factor, a TGF-beta inhibitor, a notch pathway inhibitor, a hepatocyte growth factor, dexamethasone, and Oncostatin M.

These and other features, objects, and advantages of the present invention will become better understood from the description that follows. In the description, reference is made to the accompanying drawings, which form a part hereof and in which there is shown by way of illustration, not limitation, embodiments of the invention. The description of preferred embodiments is not intended to limit the invention to cover all modifications, equivalents, and alternatives. Reference should therefore be made to the claims recited herein for interpreting the scope of the invention.

### BRIEF DESCRIPTION OF DRAWINGS

The disclosure will be better understood and features, aspects, and advantages other than those set forth above will become apparent when consideration is given to the following detailed description thereof. Such detailed description refers to the following drawings.

FIG. 1A-FIG. 1C show expression of human embryonic stem cell-derived hepatocytes using the methods disclosed herein. Specifically, FIG. 1 shows phase (FIG. 1A), albumin (GFP) (FIG. 1B) and AFP (tdTomato) expression (FIG. 1C) of the derived hepatocytes. High concurrent expression of both albumin and alpha-fetoprotein (AFP) indicative of late fetal liver was observed.

FIG. 2A-FIG. 2C show albumin positive colonies that did not express AFP after culturing hepatocytes on Mouse Embryonic Fibroblasts (MEFs) in SHPC medium indicating their mature nature.

FIG. 3 shows HPCs derived using the methods disclosed herein displayed typical HPC morphology and expressed the mature marker CD45 (FITC channel) but did not express the early marker CD43.

FIG. 4A-FIG. 4B show the difference in morphology of human umbilical cord vein endothelial cells (HUVEC) cultured on vitronectin (FIG. 4A) and MEFs (FIG. 4B).

FIG. 5A-FIG. 5B shows FACs analysis of the HUVEC cultured on vitronectin and MEFs. FIG. 5A shows HPC islands. FIG. 5B shows a FACs plot wherein 70% of the floater cells were CD45 positive.

FIG. 6A-FIG. 6D show that macrophages derived using the methods disclosed herein displayed typical morphology with lysosomes. FIG. 6A shows attached macrophage derived from HPCs differentiated from HUVEC. FIG. 6B shows macrophages in suspension differentiated from HUVEC. FIG. 6C shows macrophages in suspension differentiated from primary human liver sinusoidal endothelial cells (PHLSEC). FIG. 6D shows macrophages in suspension differentiated from human primary arterial endothelial cells (HPAEC)-derived HPCs.

FIG. 7A-FIG. 7B show a typical macrophage (HUVEC-derived) that has phagocytosed RFP conjugated zymosan particles.

FIG. 8A-FIG. 8C show zymosan uptake by macrophages differentiated from HUVEC (FIG. 8A), PHLSEC (FIG. 8B), and HPAEC-derived HPCs (FIG. 8C).

### DETAILED DESCRIPTION OF THE DISCLOSURE

All publications, including but not limited to patents and patent applications, cited in this specification are herein incorporated by reference as though set forth in their entirety in the present application.

As utilized in accordance with the present disclosure, unless otherwise indicated, all technical and scientific terms shall be understood to have the meaning commonly understood by one of ordinary skill in the art. Unless otherwise required by context, singular terms shall include the plural and plural terms shall include the singular.

This disclosure relates generally to methods for generating small hepatocyte progenitor cells and hematopoietic progenitor cells from human embryonic stem cells, populations of small hepatocyte progenitor cells and hematopoietic progenitor cells, and uses thereof. The methods and population of cells disclosed herein advantageously mimic a late fetal liver undergoing hematopoiesis.

In particular embodiments provided herein is a method for obtaining small hepatocyte progenitor cells and hematopoietic progenitor cells, the method comprising: culturing human embryonic stem cells in the presence of embryonic fibroblasts in a culture medium that comprises at least one of Activin A, a bone morphogenetic protein, a fibroblast growth factor, a TGF-beta inhibitor, a notch pathway inhibitor, a hepatocyte growth factor, whereby a cell population comprising small hepatocyte progenitor cells and hematopoietic progenitor cells is obtained.

As used herein, “small hepatocyte progenitor cells (SHPCs)” are mature hepatocyte precursor cells. SHPCs have a small round morphology with clear nuclei resembling mature adult hepatocytes but smaller in size. SHPCs can originate by partial de-differentiation from mature hepatocytes when needed, such as upon liver injury or disease, to proliferate and restore liver mass.

As used herein, “hematopoietic progenitor cells (HPCs)” refers to cells present in blood and bone marrow. HPCs are essential for giving rise to blood cells. HPCs can be used for treatment of cancer and other immune system disorders.

As used herein, the term “embryonic stem cells” or “ESCs” means a pluripotent cell or population of pluripotent cells derived from an inner cell mass of a blastocyst. See Thomson et al., Science 282:1145-1147 (1998). These cells express Oct-4, SSEA-3, SSEA-4, TRA-1-60, and TRA-1-81, and appear as compact colonies having a high nucleus to cytoplasm ratio and prominent nucleolus. ESCs are commercially available from sources such as WiCell Research Institute (Madison, Wis.).

In some cases, the human ESCs were cultured in medium that further comprises dexamethasone, Oncostatin M, or combinations thereof in amounts effective and for lengths of time sufficient to direct differentiation of ESCs to SHPCs and HPCs. ESCs were cultured in the culture medium for about 18 days (e.g., 16 days, 17 days, 18 days, 20 days). In some embodiments, human ESCs were co-cultured with fibroblasts in culture medium comprising or consisting essentially of Activin A, a bone morphogenetic protein, a fibroblast growth factor, a TGF-beta inhibitor, a notch pathway inhibitor, a hepatocyte growth factor, dexamethasone, and Oncostatin M, for about 18 days. In some embodiments the culture medium comprises or consists essentially of 100 ng/ml Activin A, about 20 ng/ml bone morphogenetic protein, about 25 ng/ml fibroblast growth factor, about 10 μM TGF-beta inhibitor, about 10 μM notch pathway inhibitor, about 20 ng/ml hepatocyte growth factor, about 10 ng/ml dexamethasone, and about 0.1 μM Oncostatin M. In some embodiments, the embryonic fibroblasts were mouse embryonic fibroblasts (MEFs). The culturing can take place on any appropriate surface (e.g., in two-dimensional or three-dimensional culture).

In some embodiments, the bone morphogenetic protein (BMP) was bone morphogenetic protein 2. In some embodiments, the fibroblast growth factor was fibroblast growth factor 4. In some embodiments, the TGF-beta inhibitor was SB 431542. In some embodiments, the notch pathway inhibitor was (2S)-N-[(3,5-Difluorophenyl)acetyl]-L-alanyl-2-phenyl]glycine 1,1-dimethylethyl ester (DAPT).

In particular embodiments, the Activin A, a bone morphogenetic protein, a fibroblast growth factor, a TGF-beta inhibitor, a notch pathway inhibitor, a hepatocyte growth factor, dexamethasone, and Oncostatin M were added to the culture medium over a span of 18 days. For example, Activin A was added to the culture medium for the first 3 days at a concentration of about 100 ng/ml. Then for the next 5 days, growth factors, bone morphogenetic protein 2 (BMP2) and fibroblast growth factor 4 (FGF4) were added to the culture medium in combination with the TGF-beta inhibitor, SB 431542, and notch pathway inhibitor, DAPT. Further, during the 5-day period, BMP2 was added at a concentration of about 20 ng/ml, FGF4 was added at a concentration of about 25 ng/ml, SB 431542 was added at a concentration of about 10 μM, and DAPT was added at a concentration of about 10 μM. For the following 10 days, hepatocyte growth factor (HGF), Oncostatin M, dexamethasone, SB 431542, and DAPT, or combinations thereof, were added to the culture medium. Further, during the 10-day period, HGF was added at a concentration of about 20 ng/ml, Oncostatin M was added at a concentration of about 10 ng/ml, dexamethasone was added at a concentration of about 0.1 μM, SB 431542 was added at a concentration of about 10 μM, and DAPT was added at concentration of about 10 μM.

In a further embodiment, a substantially pure population of small hepatocyte progenitor cells and CD45⁺ hematopoietic progenitor cells were isolated using the methods disclosed herein. In another embodiment, a pharmaceutical composition was comprised of the isolated small hepatocyte progenitor cells and CD45⁺ hematopoietic progenitor cells and a pharmaceutically acceptable carrier.

In some embodiments provided herein was a method for culturing human primary endothelial cells in the presence of embryonic fibroblasts in a culture medium whereby a cell population comprising CD45⁺ hematopoietic progenitor cells was obtained. In particular embodiments, the culture medium was from Lonza (Catalog# CC 3162 comprising EBM-2 basal medium (CC-3156) supplemented with FBS, Hydrocortisone, hFGF-B, VEGF, R3-IGF-1, Ascorbic acid, hEGF, GA-1000 and Heparin. Other suitable culture mediums known in the art can be used in the methods disclosed herein.

The human primary endothelial cells used in the methods disclosed herein can be human umbilical vein endothelial cells, primary human liver sinusoidal endothelial cells, or human primary arterial endothelial cells. Human umbilical vein endothelial cells (HUVECs) were cells derived from the endothelium of veins from the umbilical cord. Primary Human Liver Sinusoidal Endothelial Cells (PHLSECs) were isolated from human liver tissue. Human primary arterial endothelial cells (HPAECs) were derived from arteries and human primary venous endothelial cells (HPVECs) were isolated from veins.

In a further embodiment, a substantially pure population of CD45⁺ hematopoietic progenitor cells were isolated using the methods disclosed herein. In another embodiment, a pharmaceutical composition was comprised of the isolated CD45⁺ hematopoietic progenitor cells and a pharmaceutically acceptable carrier.

The fibroblasts used in co-culture with the small hepatocyte progenitor cells and/or hematopoietic progenitor cells may be human embryonic fibroblasts (Kibschuli et al., 2011, Stem Cell Res. 6:70-82) or mouse embryonic fibroblasts. Mouse embryonic fibroblasts or human embryonic fibroblasts may be obtained from any suitable source.

For several of the biological markers described herein, expression will be low or intermediate in level. While it is commonplace to refer to cells as “positive” or “negative” for a particular marker, actual expression levels are a quantitative trait. The number of molecules on the cell surface can vary by several logs, yet still be characterized as “positive.” Accordingly, characterization of the level of staining permits subtle distinctions between cell populations. Expression levels can be detected or monitored by flow cytometry, where lasers detect the quantitative levels of fluorochrome (which is proportional to the amount of cell surface antigen bound by the antibodies). Flow cytometry or fluorescence-activated cell sorting (FACS) can be used to separate cell populations based on the intensity of antibody staining, as well as other parameters such as cell size and light scatter. Although the absolute level of staining can differ with a particular fluorochrome and antibody preparation, the data can be normalized to a control.

Any appropriate method can be used to detect expression of biological markers characteristic of cell types described herein. For example, the presence or absence of one or more biological markers can be detected using, for example, RNA sequencing (e.g., RNA-seq), immunohistochemistry, polymerase chain reaction, quantitative real time PCR (qRT-PCR), or other technique that detects or measures gene expression. RNA-seq is a high-throughput sequencing technology that provides a genome-wide assessment of the RNA content of an organism, tissue, or cell. Alternatively, or additionally, one may detect the presence or absence of, or measure the level of, one or more biological markers of SHPCs and HPCs using, for example, Fluorescence in situ Hybridization (FISH; see WO98/45479 published October, 1998), Southern blotting, Northern blotting, or polymerase chain reaction (PCR) techniques, such as qRT-PCR. In some embodiments, a cell population obtained according to a method provided herein was evaluated for expression (or the absence thereof) of biological markers of SHPCs such as CD44. In some embodiments, SHPCs express the small hepatocyte progenitor cell markers CD44 and one or more of the hepatocyte markers albumin (ALB), alpha-fetoprotein, SERPINAL CYP2E1, CYP3A5, CYP1A1, CYP1B1, UGT1A1, UGT1A6, and UGT1A9. In some embodiments, a cell population obtained according to a method provided herein was evaluated for expression (or the absence thereof) of biological markers of HPCs such as CD45 and CD43. “CD45” is a late fetal marker, and “CD43” is an early fetal marker. In some embodiments, the HPCs are CD45⁺. In some embodiments, the HPCs do not express CD43. Quantitative methods for evaluating expression of markers at the protein level in cell populations are also known in the art. For example, flow cytometry is used to determine the fraction of cells in a given cell population that express or do not express biological markers of interest.

In particular embodiments are provided methods for obtaining a population of macrophages, the method comprising: culturing the population of comprising CD45⁺ hematopoietic progenitor cells obtained according to the methods disclosed herein in absence of a matrix in a culture medium and under conditions suitable for generation of macrophages, whereby a cell population comprising macrophages was obtained. In particular embodiments, the culture medium was DMS, as described in Swartz et al. (2015, Proc. Natl. Acad. Sci. USA 112: 12516-21). Other suitable culture mediums known in the art can also be used in the methods disclosed herein.

In some embodiments provided herein was a cell culture medium comprising Activin A, a bone morphogenetic protein, a fibroblast growth factor, a TGF-beta inhibitor, a notch pathway inhibitor, a hepatocyte growth factor, dexamethasone, and Oncostatin M. In some embodiments, the bone morphogenetic protein (BMP) was bone morphogenetic protein 2, the fibroblast growth factor was fibroblast growth factor 4, the TGF-beta inhibitor was SB 431542 and the notch pathway inhibitor was DAPT.

In some embodiments, the cell culture comprises between about 75 ng/ml and about 125 ng/ml Activin A, between about 10 ng/ml and about 30 ng/ml bone morphogenetic protein, between about 15 ng /ml and about 35 ng/ml fibroblast growth factor, between about 5 μM and about 15 μM TGF-beta inhibitor, between about 5 μM and about 15 μM notch pathway inhibitor, between about 10 ng/ml and about 30 ng/ml hepatocyte growth factor, between about 5 ng/ml and about 15 ng/ml dexamethasone, and between about 0.1 μM and about 0.5 μM Oncostatin M.

In some embodiments, the cell culture comprises about 100 ng/ml Activin A, about 20 ng/ml bone morphogenetic protein, about 25 ng/ml fibroblast growth factor, about 10 μM TGF-beta inhibitor, about 10 μM notch pathway inhibitor, about 20 ng/ml hepatocyte growth factor, about 10 ng/ml dexamethasone, and about 0.1 μM Oncostatin M.

The disclosed methods for obtaining hepatocytes and hematopoietic progenitors from human ESCs and hematopoietic progenitors from human primary endothelial cells provide necessary tools for various aspects of drug and toxicity testing of liver diseases. Unlike other strategies for co-differentiating hepatocytes and hematopoietic progenitors, this approach does not require primary human hepatocytes (PHHs) which de-differentiate in culture and cannot be passaged, making this a more feasible approach for laboratory research. The technology can advantageously produce functional hematopoietic progenitor cells from both ES and primary human endothelial cells, displaying late fetal markers.

Various exemplary embodiments of compositions and methods according to this invention are now described in the following non-limiting Examples. The Examples are offered for illustrative purposes only and are not intended to limit the scope of the present invention in any way. Indeed, various modifications of the invention in addition to those shown and described herein will become apparent to those skilled in the art from the foregoing description and the following examples and fall within the scope of the appended claims.

### EXAMPLES

The Examples that follow are illustrative of specific embodiments of the disclosure, and various uses thereof. They are set forth for explanatory purposes only, and should not be construed as limiting the scope of the disclosure in any way.

### Example 1: Derivation of SHPC and HPCs from ES Cells

### Primary Embryonic Stem Cells

Primary human embryonic stem cells (H9) used herein were isolated by one of the inventors, James Thomson (PMID: 9804556).

### MEF Culture

Mouse embryonic fibroblasts (MEFs) were obtained from E13.5 embryos of pregnant CD-1 female mice (purchased from Charles River Laboratories) 13 days post plugging, where plugging day was considered day 0.5. MEFs were cultured from passage 1 (pl) to passage 3 (p3) in growth medium (1×DMEM, 10× heat inactivated fetal bovine serum (FBS), 1× nonessential amino acids) and irradiated with a dose of 80Gy using a Mark I 137Cs irradiator. Irradiated MEFs were seeded onto 0.1% gelatin coated plates at a concentration of 1.8×10⁵ cells/mL to be used for culturing SHPCs and HPCs.

### SHPC and HPC Derivation and Culture

Differentiation using a H9 ES cell-derived dual reporter clone where GFP was driven by the mature hepatic gene albumin and tdTomato driven by the promoter of the fetal gene alpha-fetoprotein (AFP) was performed. Primary singularized ES cells were cultured on a top feeder layer of MEFs (3×10\hat{ }6/sq cm) in SHPC medium. SHPC medium was E6 medium (DMEM/F12 medium, L-ascorbic acid-2-phosphate magnesium (64 mg/l); sodium selenium (14 μg/l); insulin (20 mg/l); NaHCO₃ (543 mg/l); and transferrin (10.7 mg/1), Chen et al., Nature Methods 2011 8(5):424-9) supplemented with Activin A, BMP2, FGF4, SB 431542, DAPT, Dexamethasone (1 μM), HGF, and Oncostatin M. For the first 3 days, cells were treated with 100 ng/ml activin A. For the next five days the cells were treated with 20 ng/ml BMP2, 25 ng/ml FGF4 and 10 μM SB 431542 and 10 μM DAPT. For the next ten days, the cells were treated with 20 ng/ml HGF, 10 ng/ml Oncostatin M, 0.1 μM dexamethasone and 10 μM SB 431542 and 10 μM DAPT.

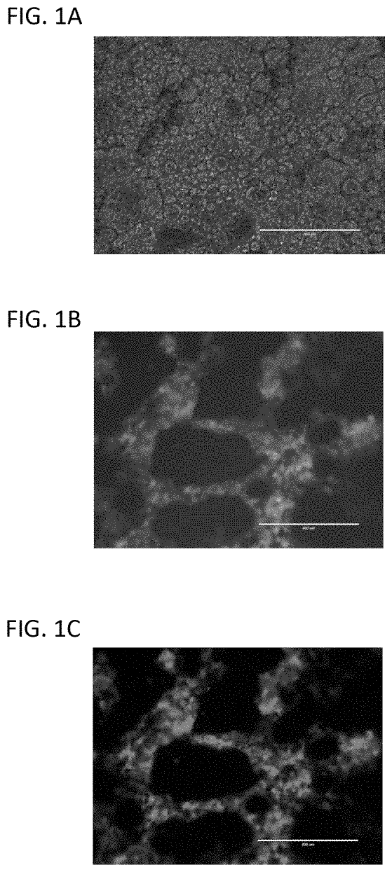

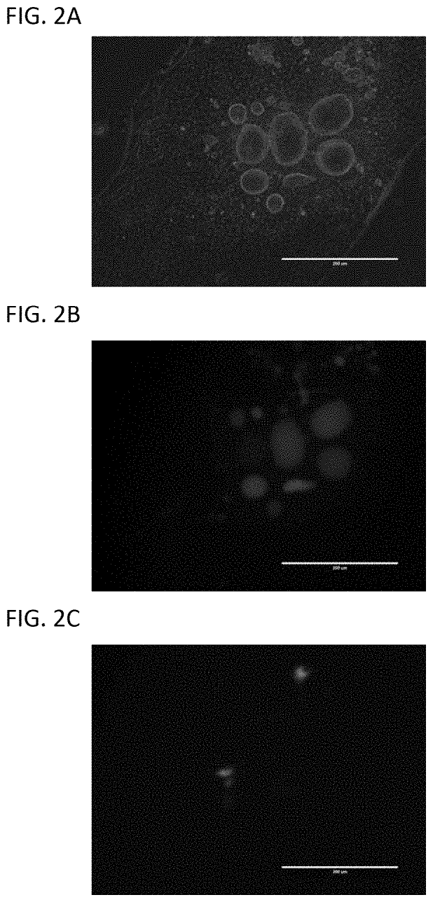

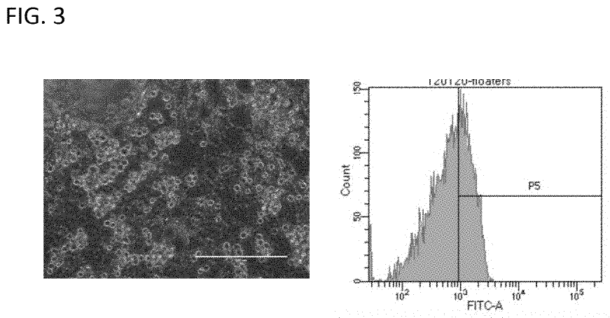

FIGS. 1A through 1C show phase, albumin (GFP) and AFP (tdTomato) expression of the derived hepatocytes. High concurrent expression of both albumin and AFP, indicative of late fetal liver, was observed. Upon culturing these hepatocytes on MEFs in SHPC medium, Sengupta et al., 2020, Research in Toxicology 1: 70-84, albumin positive colonies appeared that did not express AFP (FIGS. 2A through 2C) indicating their mature nature. Derivation of hepatocytes on MEFs concurrently gives rise to hematopoietic progenitor cells (HPCs). FIG. 3 shows these cells displayed typical HPC morphology and expressed the mature marker CD45 (FITC channel) and did not express the early marker CD43.

SHPCs and HPCs were passaged by dissociation with 10 × Trypsin-EDTA (Sigma-Aldrich) and split 1:2 every 3 days with daily feeding. The SHPCs and HPCs were not sorted from the MEFs during passaging. After trypsinization for 5 min in a 37 ° C. incubator, trypsin was neutralized with SHPC medium. Next, all the cells (SHPC, HPCs, MEFs) were pipetted up and down and transferred to a 50 ml centrifuge tube and allowed to settle for 5 min. The supernatant containing mostly SHPCs and HPCs were then seeded on new MEF plates leaving majority of the feeder cells at the bottom of the centrifuge tube. Importantly, MEFs were plated 24 h prior to SHPC and HPC seeding.

### Example 2: Derivation of HPCs from Endothelial Cells and Differentiation to Macrophages

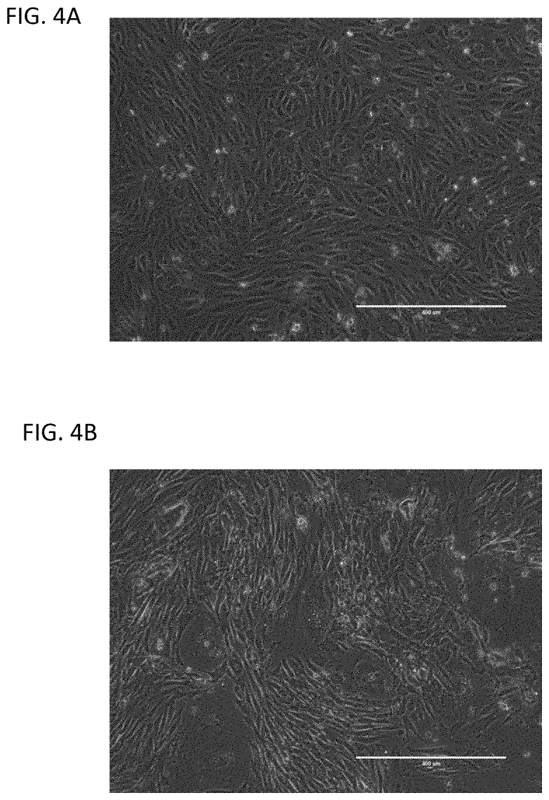

The derivation of HPCs from primary human endothelial cells is demonstrated in this example. Human umbilical cord vein endothelial cells (HUVEC) were cultured in ECM (endothelial cell medium) from Lonza (Catalog #: CC-3162). The medium comprises EBM-2 basal medium (CC-3156) supplemented with FBS, Hydrocortisone, hFGF-B, VEGF, R3-IGF-1, Ascorbic acid, hEGF, GA-1000 and Heparin. HUVEC cells displayed tubular structures when grown on MEFs but did not demonstrate the same structures when grown on vitronectin. This difference in morphology of HUVEC was shown in FIG. 4. Upon reaching confluency, the endothelial cells formed island like structures from which HPCs bud off (FIGS. 4A and 4B). HPCs were generated only when HUVECs (and other endothelial cells) were cultured on MEFs only, and not on vitronectin which was used traditionally to grow endothelial cells. HPCs will also arise if a culture medium other than Lonza's was used (from other vendors), however, MEF's were necessary for formation of HPCs from endothelial cells.

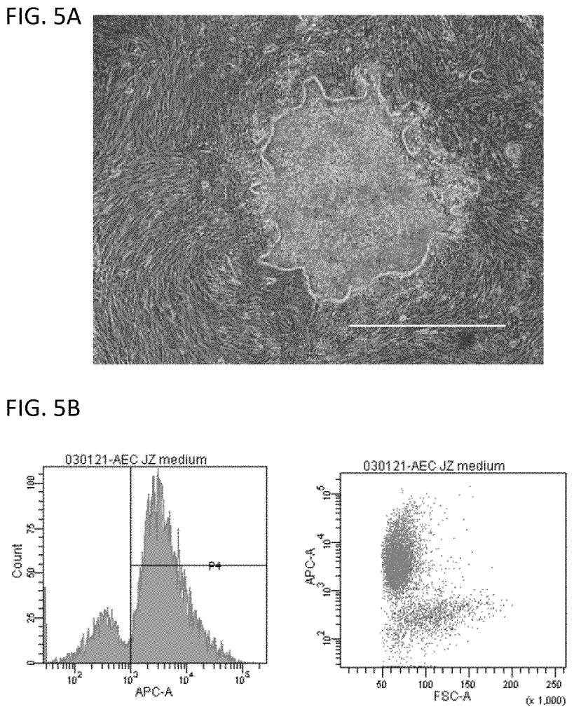

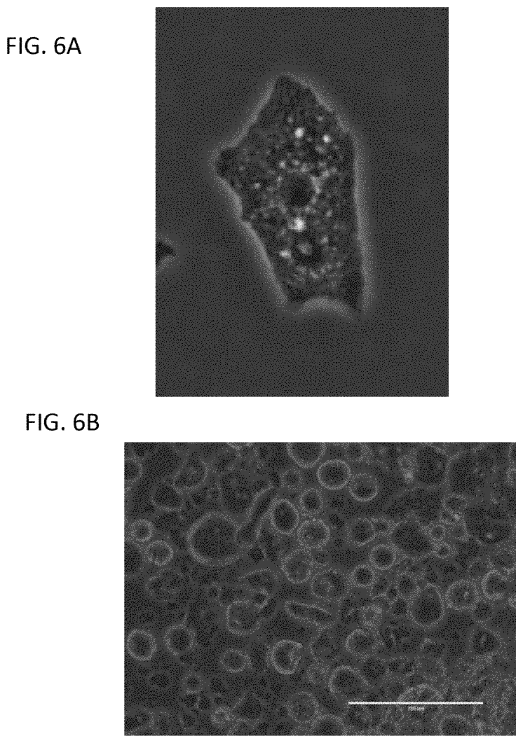

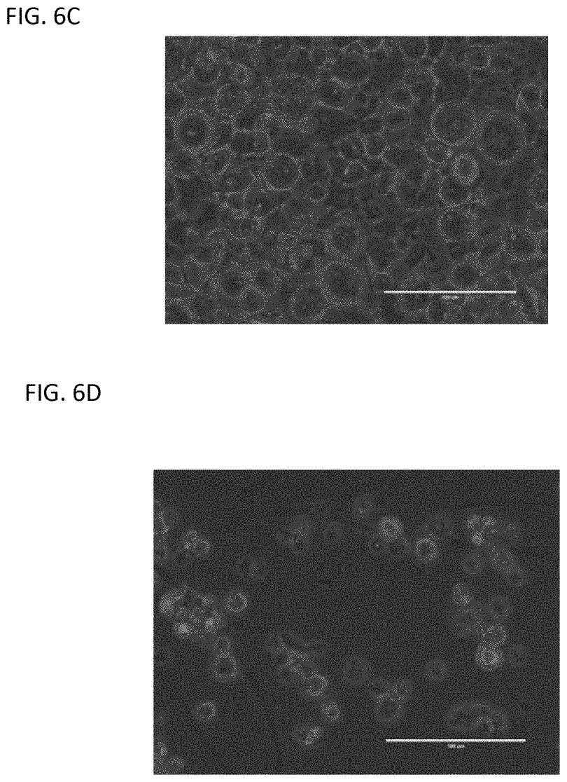

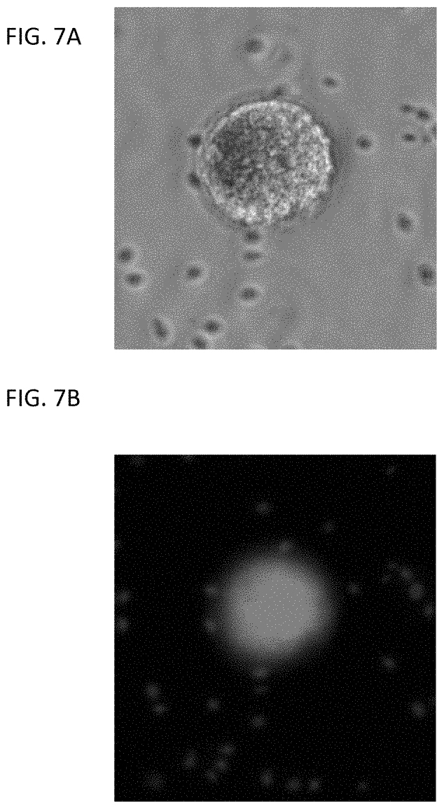

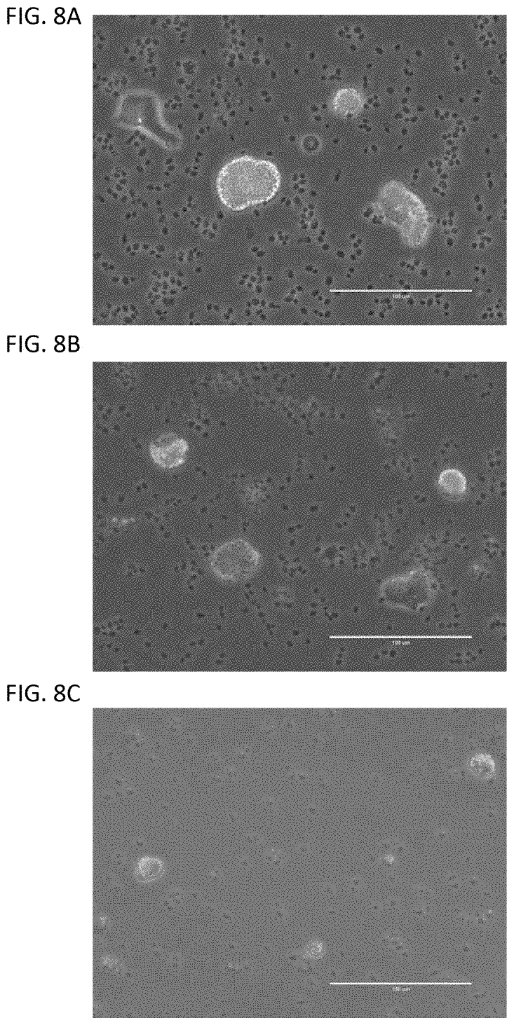

These cells then floated up and FACs analysis showed 70% of these cells were CD45 positive (FIGS. 5A and 5B). As seen from the FACs plot, the floaters were small (FIGS. 5A and 5B), which is typical of HPCS. To confirm that human primary endothelial cells can give rise to HPCs (and not HUVEC), human liver sinusoidal endothelial cells (PHLSECs), human primary venous endothelial cells (HPVECs), or human primary arterial endothelial cells (HPAECs) were grown and similar results were observed. Next, HUVEC, PHLSEC, HPVEC and HPAEC-derived CD45 cells were differentiated to the macrophage lineage. Cells were differentiated on bare plastic dishes without any matrix in a cell culture medium, DM5, as described in Swartz et al., PNAS (PMID: 26392547) and under conditions suitable for generation of macrophages, whereby a cell population comprising macrophages was obtained. The differentiated macrophages were loosely attached and most macrophages floated up, but the attached cells displayed typical macrophage morphology with lysosomes (FIGS. 6A through 6D). FIGS. 7A and 7B shows a typical macrophage (HUVEC-derived) that has phagocytosed RFP conjugated zymosan particles. The assay was performed on all three cell-type derived macrophages (FIGS. 8A through 8C) and showed that HUVEC, PHLSEC and HPAEC-derived macrophages efficiently took up zymosan particles.

All publications, patents, and patent applications mentioned in this specification are herein incorporated by reference to the same extent as if each individual publication, patent, and patent application was specifically and individually indicated to be incorporated by reference.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings the disclosure, and the appended claims. The mere fact that certain measures or features are recited in mutually different dependent claims does not indicate that the combination of these measures or features cannot be used. Any reference signs in the claims should not be construed as limiting the scope.

What is claimed is:

1. A method for obtaining small hepatocyte progenitor cells and hematopoietic progenitor cells, the method comprising:
culturing human embryonic stem cells in the presence of embryonic fibroblasts in a culture medium that comprises at least one of Activin A, a bone morphogenetic protein, a fibroblast growth factor, a TGF-beta inhibitor, a notch pathway inhibitor, a hepatocyte growth factor,
whereby a cell population comprising small hepatocyte progenitor cells and hematopoietic progenitor cells is obtained.

2. The method of claim 1, wherein the bone morphogenetic protein (BMP) is bone morphogenetic protein 2.

3. The method of claim 1, wherein the fibroblast growth factor is fibroblast growth factor 4.

4. The method of claim 1, wherein the TGF-beta inhibitor is SB 431542.

5. The method of claim 1, wherein the notch pathway inhibitor is DAPT.

6. The method of claim 1, wherein the culture medium further comprises dexamethasone, Oncostatin M, or combinations thereof.

7. The method of claim 1, wherein the embryonic fibroblasts are mouse embryonic fibroblasts or human embryonic fibroblasts.

8. The method of claim 1, wherein the embryonic stem cells are cultured for about 18 days.

9. The method of claim 1, wherein the hematopoietic progenitor cells are CD45⁺.

10. The method of claim 9, wherein the hematopoietic progenitor cells do not express CD43.

11. A substantially pure, isolated population of small hepatocyte progenitor cells and CD45⁺ hematopoietic progenitor cells obtained according to the method of claim 1.

12. A pharmaceutical composition comprising the cell population of claim 11 and a pharmaceutically acceptable carrier.

13. A method for obtaining hematopoietic progenitor cells, the method comprising:
culturing human primary endothelial cells in the presence of embryonic fibroblasts in a culture medium whereby a cell population comprising CD45⁺ hematopoietic progenitor cells is obtained.

14. The method of claim 13, wherein the endothelial cells are human umbilical cord vein endothelial cells (HUVEC,) human liver sinusoidal endothelial cells (PHLSECs), human primary venous endothelial cells (HPVECs), or human primary arterial endothelial cells (HPAECs).

15. The method of claim 13, wherein the embryonic fibroblasts are mouse embryonic fibroblasts or human embryonic fibroblasts.

16. A substantially pure, isolated cell population of CD45⁺ hematopoietic progenitor cells obtained according to the method of claim 13.

17. A pharmaceutical composition comprising the cell population of claim 16 and a pharmaceutically acceptable carrier.

18. A method for obtaining a population of macrophages, the method comprising:
culturing the population of comprising CD45⁺ hematopoietic progenitor cells obtained according to the method of claim 13 in absence of a matrix in a culture medium and under conditions suitable for generation of macrophages, whereby a cell population comprising macrophages is obtained.

19. A cell culture medium comprising Activin A, a bone morphogenetic protein, a fibroblast growth factor, a TGF-beta inhibitor, a notch pathway inhibitor, a hepatocyte growth factor, dexamethasone, and Oncostatin M.

20. The cell culture of claim 19, wherein the bone morphogenetic protein (BMP) is bone morphogenetic protein 2, the fibroblast growth factor is fibroblast growth factor 4, the TGF-beta inhibitor is SB 431542 and the notch pathway inhibitor is DAPT.

21. The cell culture of claim 19, comprising between about 75 ng/ml and about 125 ng/ml Activin A, between about 10 ng/ml and about 30 ng/ml bone morphogenetic protein, between about 15 ng /ml and about 35 ng/ml fibroblast growth factor, between about 5 μM and about 15 μM TGF-beta inhibitor, between about 5 μM and about 15 μM notch pathway inhibitor, between about 10 ng/ml and about 30 ng/ml hepatocyte growth factor, between about 5 ng/ml and about 15 ng/ml dexamethasone, and between about 0.1 μM and about 0.5 μM Oncostatin M.

22. The cell culture of claim 19, comprising about 100 ng/ml Activin A, about 20 ng/ml bone morphogenetic protein, about 25 ng/ml fibroblast growth factor, about 10 μM TGF-beta inhibitor, about 10 μM notch pathway inhibitor, about 20 ng/ml hepatocyte growth factor, about 10 ng/ml dexamethasone, and about 0.1 μM Oncostatin M.
